(19) United States
(12) Patent Application Publication
(10) Pub. No.: US 2008/0131311 A1
(43) Pub. Date: Jun. 5, 2008

(54) Fluorescent lamp device capable of cleaning air

(76) Inventor: Wei-Hong Wang, Tao Yuan City (TW)

Correspondence Address: BIRCH STEWART KOLASCH & BIRCH, PO BOX 747, FALLS CHURCH, VA, 22040-0747, US

(21) Appl. No.: 11/976,050

(22) Filed: Oct. 19, 2007

Related U.S. Application Data
(62) Division of application No. 10/757,463, filed on Jan. 15, 2004.

(30) Foreign Application Priority Data
May 16, 2003 (TW) 92113409

(51) Int. Cl. A61L 9/00

(52) U.S. Cl. 422/4

(57) ABSTRACT

An air cleaning fluorescent lamp is designed and prepared by coating photocatalysis materials as nano-crystalline TiO₂ anatase or like as sol with some additive, made by sol-gel techniques on a glass fiber cloth or glass fiber sleeve to be acted upon under visible light, and then wrapping the cloth or placing the sleeve on a fluorescent lamp. When the lamp is lighted, white light is not only used for illumination, but also used to clean the air, as fluorescence radiates on the surface of the photocatalysis materials to generate free electron and electron hole pairs that will activate as the waste has is decomposed, to clean the air and provide self cleaning.

This application is a Divisional of co-pending application Ser. No. 10/757,463, filed on Jan. 15, 2004, and for which priority is claimed under 35 USC §120; and this application claims priority of Application No. 092113409 filed in Taiwan, R.O.C. on May 16, 2003 under 35 USC §119; the entire contents of all of which are hereby incorporated by reference.

### BACKGROUND OF THE INVENTION

1. Field of the Invention

The present invention relates to a fluorescent lamp devices capable of cleaning air, and more particularly, to a fluorescent lamp wrapped with glass fiber cloth coated with anatase TiO₂ nano-crystalline sol, which is a photo-catalytic material can be acted under fluorescent lamp with visible light.

The present invention also discloses a method comprising the steps of preparing semiconductor nano-crystalline anatase TiO₂ sol using titanium alkoxide Ti(OR)₄ as a main component in combination with chelating agents in aqueous solution. The thus-formed photo-catalytic coating glass fiber cloth and glass fiber sleeve covering or wearing the fluorescent lamp tube can be tailored into the shape of the lamp tube. The present invention adopts various fluorescent lamps having visible fluorescence light with small amount or without of 365 nm and 405 nm near UV light, thereby forming an air cleaning fluorescent lamp, which provide lighting and air cleaning functions. The present invention method for fabricating a fluorescent lamp which is designed and fabricated, based on sol-gel coating techniques, used to clean the air around the lamp to the environment. The photo-catalytic materials in the sol, can be acted under the visible light, comprising anatase TiO₂ as the main component, and/or semiconductor impregnated with precious metal as Au, Pd or Pt, more and/or doped with transition metal oxide such as WO₃, ZnO, SnO₂, or Fe₂O₃, and/or substituted the oxygen of the titanium oxide with N, S, P, or F element by thermal diffusion or chemical reaction, must be in the nano-crystalline size, coated on a glass-fiber-cloth or sleeve.

2. Description of the Related Art

Sol-gel techniques have been emphasized today by technically advanced countries. When developments of traditional chemical and physical technologies have met bottlenecks, and in particular, when inorganic materials produced through traditional techniques no longer satisfy requirements, in particular for thin film coating, materials having multiple components and special structures that cannot processed by conventional physical and/or chemical method, as well as when coating those material on irregularly curve surfaces cannot been achieved by conventional evaporating disposition techniques, the sol-gel technique, can easily generate a metal oxide film thereon. At the same time, it is the characteristic feature of the sol-gel technique that the photo-catalyst film obtained thereby has a porous crystallite structure required by the photo-catalysis reaction. Therefore, sol-gel coating techniques have become one of the most interesting techniques for research and development in the latter part of the twentieth century.

Recently, preparation of catalysts by sol-gel techniques has also received emphasis by chemical industries. In particular, photo-catalytic techniques are the most important of these, including the early developed photo-catalytic powders for treating waste water, as described in, for example: Robat A. Clyde, U.S. Pat. No. 4,446,236; Robat E. Hetrick, Ford Motor Company, U.S. Pat. No. 4,544,470; Yashiaki Harada et al., Osaka Gas Company, U.S. Pat. No. 4,699,720; Tomoji Kawai, et al., Nomura Micro Science Co., U.S. Pat. No. 4,863,608; David G. Ritchie, U.S. Pat. No. 5,069,885; Gerald Cooper, et al., Photo Catalytics Inc., U.S. Pat. Nos. 5,116,582; 5,118,422; 5,174,877; and 5,294,315; Adam Heller, et al., Board of Regents, The University of Texas System, U.S. Pat. No. 5,256,616; Ali Safarzedeh-Amiri, Cryptonics Corporation, U.S. Pat. No. 5,266,214; Fausto Miano & Borgarello, Eniricerche S.p.a., U.S. Pat. No. 5,275,741; Nancy S. Foster et al., Regents of the University of Colorado, U.S. Pat. No. 5,332,508; Ivan Wlassics et al., Ausimont S.p.a., U.S. Pat. No. 5,382,337; Paul C. Melanson & James A. Valdez, Anatol Corporation, U.S. Pat. No. 5,395,522; Henry G. Peebles III et al., American Energy Technology, Inc., U.S. Pat. No. 5,449,466; Brain E. Butters & Anthony L. Powell, Purific Environmental Technologies, Inc., U.S. Pat. Nos. 5,462,674; 5,554,300; and 5,589,078; Yin Zhang, et al., Board of Control of Michigan Technology University, U.S. Pat. No. 5,501,801; Clovis A. Linkous, University of Central Florida, U.S. Pat. No. 5,518,992; and Eiji Normura & Tokuo Suita, Ishihara Sanyo Kaisha Ltd., U.S. Pat. No. 5,541,096.

The above-mentioned U.S. patents relate chiefly to water treatment, in which, the case of granular catalysts, a filtration recovering apparatus is invariably used, and it is of the most importance that such photo-catalysis needs sufficient dissolved oxygen in water. Otherwise, an aerating operation must be carried out for supplying oxygen required by the photo-catalytic degradation.

Since then, photo-catalysts have also been used for treating waste gases, such as those described in, for example, Gregory B. Roupp & Lynette A. Dibble, Arizona State University, U.S. Pat. No. 5,045,288; Jeffrey g. Sczechowski et al., The University of Colorado, U.S. Pat. No. 5,439,652; William A. Jacoby & Danial M. Blake, U.S. Pat. No. 5,449,443; Zhenyyu Zhang & James R. Gehlner, Inrad, U.S. Pat. No. 5,468,699; and Franz D. Oeste, Olga Dietrich Neeleye, U.S. Pat. No. 5,480,524.

The above-mentioned patents relate originally to treatment of waste gases, and basically, were carried out in a closed reactor. Utilization or operation of granular catalysts or catalysts coating granules is usually needed with complicated equipment.

The above-described disadvantages made the prior art photo-catalysts difficult to apply to treatment of polluted air in our living environment, and among them, the only waste water and/or waste gases disposal photo-catalytic reactor comprising a UV lamp wrapped with photocatalyst coated film having fibers as supports thereof was the one described in Michael K. Robertson & Robert G. Henderson, Neutech Energy Systems Inc., U.S. Pat. No. 4,982,712. As mentioned above, such a reactor was of a closed type such that counter-flow of gases had to be forced by a blower, making such a reaction system impractical for use in living place.

A UV lamp for air cleaning, generally relies on the sustained oxidative degradation of organic and/or inorganic hazardous materials in the air by a photo-catalytic reaction to render them into benign substances such as water or carbon dioxide. For example, Hiroshi Taoda and Watanabe, U.S. Pat. No. 5,650,126 and U.S. Pat. No. 5,670,206, which coated on the surface of UV lamp with TiO₂ materials, than heating and anneal to TiO₂ Anatase film. Also in U.S. Pat. No. 6,135,838 and U.S. Pat. No. 6,336,998, which wrapped with TiO₂ Anatase sol coating glass fiber cloth or sleeve on the UV lamp, are owned by same applicants of the present application.

Since a UV lamp is not a commercially available lighting apparatus, some research has focused on a commercial fluorescent lamp having photo-catalytic coating for cleaning air. U.S. Pat. No. 6,024,929 by Ichikawa Shinichi, Furukawa Yashinori, and Azuhata Shigeru discloses a light-transmissive and transparent film photo-catalyst made of anatase-type titanium dioxide and alpha iron oxide formed on an outside surface of a glass tube used for a fluorescent lamp. The thin film photo-catalyst is formed so that electrons and holes generated inside the film by light irradiation can migrate to the surface of the film and generate various active species at the surface of the film by contacting with the room air, enabling an excellent deodorization effect, bactericidal and fungicidal activity and contamination preventing effect. A TiO₂ gel solution made of a mixture of titanium alkoxide, acid and alcohol is used to form the thin film titanium dioxide coating, and a iron oxide gel solution made of a mixture of an iron compound, acid and alcohol is used to form the thin film alpha iron oxide coating. The temperature for baking the sol solution adhered to the outside wall of the glass tube is in a range of 450-600 degrees centigrade in the case of forming thin film anatase-type titanium oxide and is in a range of 560-770 degrees centigrade in the case of forming an alpha iron oxide. By baking the sol solution at a high temperature as in the above-mentioned ranges, decrease the porosity of photocatalyst coating and the air pollutants contact with photocatalyst, with low efficiency for air cleaning.

U.S. Pat. No. 6,242,862 by Akira Kawakatsu and Kanagawa-ken discloses a photo-catalytic coating fluorescent lamp with complex designed membrane. The membrane is formed with two parts, one is ultra fine particle of photo-catalyst to be coated within, the other uneven hole membrane which is coated on the glass surface of fluorescent lamp. For increase the photo-catalyst efficiency about this fluorescent lamp, second layer of the membrane coated on the outside with partial overlap. Concave portions of the ground layer may penetrate to the surface of the base body or a metallic oxide structural layer provided with a lot of penetrating holes be formed on the surface for air cleaning by photo-catalytic membrane. However, the anatase TiO2 ultra fine particles are obtained from a high temperature sintering process. Although the ultra fine particles are dispensed in alcoholic solvent, the hydroxyl groups on the particle surfaces are still at a low level, resulting in poor adhesion to the fluorescent lamp. For this reason, inorganic materials for enhancing adhesion such as silane coupling agent, SiO₂ sol, TiO₂ sol, or Al₂O₃ sol are needed. The inorganic materials distributed in the coating, lower the possibility of the pollutants in the air to contact with photo-catalyst and therefore the cleaning efficiency. Furthermore, to improve the adhesion of the anatase TiO2 ultra fine particles to the lamp, complex concave process is carried out, which also reduces the cleaning efficiency.

### SUMMARY OF THE INVENTION

Accordingly, the primary object of the invention is to provide a method for preparing anatase TiO₂ nano-crystalline sol. The particle size of the anatase TiO₂ nano-crystalline is below 20 nm. Since the anatase TiO₂ sol is made in water-based solution, many hydroxyl groups are present on the surface of the anatase TiO₂ nano-crystalline. The anatase TiO₂ sol-gel film is baked at low temperatures in a range of about 100-250 degrees centigrade for removing organic solvent and organic additives, thereby obtaining anatase TiO₂ coating fully with nano-scale porous. Because the particle size of the anatase TiO₂ nano-crystalline is below about 20 nm and the primary particle achieves 1.0 nm scale. Due to the characteristics of nano-scale material, the quantum effect and surface structure, the nano-scale anatase TiO₂ coating presents photocatalystic effects even in the visible light range. Further, since no high-temperature sintering is needed, the nano-scale anatase TiO₂ coating is fully porous. Diffusion of air and organic/inorganic substances through the TiO₂ coating is thus easier thereby improving the deodorization effect, bactericidal and fungicidal activity and contamination preventing effect thereof.

It is another object of the present invention is to provide a fluorescent lamp using the above-mentioned anatase TiO₂ nano-crystalline sol coating. Since the anatase TiO₂ crystal particle is in the nano-scale, the photocatalytic reaction is quantumized to lower the activation energy of free electron from conduction band energy to react with pollutants in air. This activation energy has an original maximum value of about 0.8 eV. Due to the reduction of particles, the activation energy is lower than about 0.5 eV, which means an about 0.3 eV energy shrinkage, at least. This enables the visible light photo-catalysis to be formed, which works originally under 385 nm UV light. It is evidenced that the anatase TiO₂ crystal particle formed according to the present invention can function at 425 nm or at an even lower wavelength such as 512 nm visible light.

To achieve these and other advantages and in accordance with the purposes of the present invention, as embodied and broadly described herein, the present invention provides a photo-catalytic fluorescent lamp capable of cleaning air. The photocatalytic fluorescent lamp is wrapped with glass fiber cloth or sleeve. The glass fiber cloth or sleeve is coated with semiconductor-anatase TiO₂ nano-crystalline sol and then baked at 150-250 degrees centigrade. Since the surface area of the glass fiber cloth or sleeve is much larger than the surface area of the fluorescent lamp, the total photo-catalytic active area is significantly increased by 10 times or even more. Further, since the inorganic/organic gas can directly contact with photo-catalyst under light of the fluorescent lamp, which is significantly enhanced the photo-catalyst efficiency than directly coated on the surface of the lamp. Furthermore, the photo-catalytic reaction efficiency is greatly improved according to this invention because light irradiated from the fluorescent lamp is in substantially the same direction as the air and inorganic/organic gases diffusing to the photo-catalyst sites. The illumination of the fluorescent lamp does not affected by the glass fiber cloth or sleeve since it does not absorb visible light irradiated from the fluorescent lamp.

The present invention takes advantage of a small amount of 365 nm and 405 nm near ultraviolet (UV) light and a part of blue light from the fluorescent lamp to irradiate the photo-catalyst, thereby producing free electron-hole pairs, which continuously undergo redox reactions with harmful organic or inorganic substances in the air so as to generate benign substances such as H₂O or CO₂. The present invention photo-catalytic fluorescent lamp capable of cleaning air is installed on a fluorescent lamp base seat, so that the fluorescent lamp is easily changed and the fluorescent lamp base seat can be disposed anywhere, which is convenient and economic. When the lamp is turned on, the photo-catalytic fluorescent lamp can decompose waste gases such as organic or inorganic pollutants in the air into benign gases.

The photo-catalytic reaction is most effective when using glass fiber cloth or sleeve coated with nano-crystalline photo-catalyst. This is because the electron-hole pairs generated at the surface of the photo-catalyst upon the irradiation of light recombine and release heat in microsecond, if there are no oxygen molecules or reactants diffusing from outside to the backside of the photo-catalyst coating nearby the surface of the lamp. However, precious metals such as Pd, Pt, Au, and Ag can be added into the photocatalytic coating structure to lower the excitation energy of the photo-catalyst, such that electron hole pairs can be formed when irradiating the 365 nm and 405 nm near UV light and 480 nm blue light, and lifetime of the electron hole pairs can be elongated, thereby increasing photocatalysis efficiency to decompose waste gas in the air. The photo-catalytic redox reaction is carried out under the illumination of suitable light source in the presence of oxygen, moisture, reactants, and the catalyst.

Since the effective thickness of the photo-catalyst depends on material porosity, sol-gel coating on light-transmissible substrate has an effective thickness of about 1 micrometer. Photo-catalytic materials usually adopt vacuum coating, redox coating, or precipitating coating. The vacuum coating is usually used in plate surface processing and is not practical here. Moreover, the vacuum coating cannot obtain porous catalytic structure and Anatase crystalline structure. As for precipitating coating, the photo-catalytic metal oxide precipitates on a subject to be obtained in aqueous solution. Since the bonding force between the absorbed photo-catalyst and the surface of the subject to be coated is weak, the coated catalyst usually peels off. As for redox coating, a raw material of titanium metal or titanium metal alloy is used and undergoes high-temperature oxidation treatment to form titanium dioxide film. The base material thereof is metal and is not transparent to light. Further, the coating surface is insufficient and the photo-catalytic efficiency is low.

The present invention method for fabricating a fluorescent lamp for environment air cleaning and for treating waste gases therewith is provided, designed and fabricated based on sol-gel coating techniques. A sol of photo-catalytic materials comprising anatase TiO₂ sol as the main component, and/or other semiconductor components such as WO₃, ZnO, SnO₂, or Fe₂O₃, is coated on a glass-fiber-cloth or sleeve. Then, the cloth or sleeve is baked at low temperatures and photo-catalytically activated. The activated cloth or sleeve is then wrapped or wore on a fluorescent lamp tube. The lamp treats pollutants vapor by irradiating the light there-from onto the surface of the photo-catalytic materials to generate free electron and electron hole pairs, which can decompose waste gases such as organic or inorganic pollutants in the air into benign gases.

To achieve these and other advantages and in accordance with the purposes of the present invention, as embodied and broadly described herein, the present invention provides a method for fabricating a photo-catalytic fluorescent lamp capable of cleaning air. The method comprises preparing semiconductor nano crystal anatase TiO₂ sol using titanium alkoxide Ti(OR)₄ as a main component in combination with chelating agents in aqueous solution with suspended particle size smaller than 20 nm. Since the anatase TiO₂ sol is formed from aqueous solution, the anatase TiO₂ nano crystalline made in this manner has hydroxyl groups distributed all over the particle's surface and is thus extremely active. The baking step is carried out at a low temperature in a range of about 100-250 degrees centigrade to remove organic solvents and/or organic additives. Since the nano-particle size in the anatase TiO₂ sol is smaller than about 20 nm, the primary dry anatase TiO₂ particle size achieves a scale of 1.0 nm. Due to the characteristic of such nano material, the anatase TiO₂ particle made in this manner has a photocatalytic ability even in the visible light range. Since the anatase TiO₂ sol coated on the glass fiber cloth or sleeve does not need to heat through high temperature sintering process, the resultant anatase TiO₂ coating has many nano scale pores, through which air and organic/inorganic gases diffuse inside the photo-catalytic coating film and thus get higher adsorption and photo-catalysis for air cleaning effect., and thus increase anti-microbial ability also.

Other nano particle or nano crystalline particle components such as WO₃, ZnO, SnO₂, and Fe₂O₃ are formed by dissolving organic or inorganic salts of W, Zn, Sn, and Fe in alcoholic solvent. Or, nano metal oxide particles or crystalline particles are prepared first, then dispersed in solvents to form sol. The above-mentioned nano particle or nano crystalline particle components in sol-gel form are incorporated into the anatase TiO₂ sol to form anatase TiO₂ sol mixture. Using the glass fiber woven cloth or sleeve to conduct photocatalytic sol-gel coating not only increases surface area of photocatalyst but helps waste gases in the air more easily diffuse to photocatalytic active sites. The glass fiber woven cloth or sleeve is woven by conventional weaving methods. The glass fiber diameter is between about 10-100 micrometers, with fiber in bundle at number about 1-10, and a porosity of about a 100-1000 mesh. The woven glass fiber or sleeve may be treated with silane coupling agents to strength its structure. Other materials such as quartz may be used.

The glass fiber cloth or sleeve can be impregnated in batches or continuously with the photo-catalyst sol by a roller, wherein, through controlling the drawing speed of the cloth and the humidity and temperature in the air, a uniform layer (about 0.1-1.0μ) of photocatalyst coating can he applied on the surface of the glass fiber cloth or sleeve. The coated fiber cloth or sleeve undergoes evaporation in the air for about 1-10 minutes and is baked at a temperature of about 150-250 degrees centigrade for about 10-100 minutes to produce a photo-catalyst coated glass fiber cloth or sleeve.

In the production of the above-described photo-catalyst coated glass fiber cloth or sleeve, in order to improve the efficiency of treating waste gases, it can be soaked with aqueous solution containing metal salts having oxidative catalytic action. Such metal salts include precious metals such as Pd, Pt, Au and Ag. or transition metal such as Fe, Mo, Nb, V, Ce or Cr. The glass fiber cloth or sleeve is ready for use after being soaked with oxidative catalyst and dried. The concentration of the oxidative catalyst precious metal solution is a factor of fluorescent lamp illumination efficiency. If the precious metal adhesion on the anatase TiO2 coating is larger than about 0.1 wt %, the nano metals significantly absorb visible light and thus decrease fluorescent lamp illumination efficiency.

The above-said anatase TiO₂ sol photo-catalytic coating glass fiber cloth or sleeve can be cut into a desired size to wrap the fluorescent lamp. The cut size depends on lamp length and layers when wrapping the lamp. After covering the lamp with the coated cloth, the ends and/or edges of the wrapping cloth are fixed by UV curable and resistant glue, or fixed by sawing or laser sintering. When using a longitudinally extended outer sleeve covering the fluorescent lamp, the outer sleeve has an inner diameter larger than an outer diameter of the fluorescent lamp tube. The outer sleeve has a length substantially equal to a length of the fluorescent lamp tube. The outer sleeve has opposing open ends that are thermally sealed and fixed on the lamp.

The present invention adopts various fluorescent lamps having a fluorescent visible light wavelength of 420-700 nm and small amount of 365 nm and 405 nm near UV. To allow as much visible light as possible pass through the photo-catalytic coating glass fiber cloth or sleeve, a finer or looser glass fiber cloth or sleeve substrate is used to undergo photo-catalytic sol dipping or soaking, to form a uniform and transparent photo-catalytic coating glass fiber cloth or sleeve which is wrapped on the fluorescent lamp tube or cover on the lamp tube, thereby forming an air cleaning fluorescent lamp, which provides lighting and air cleaning functions.

The photo-catalytic air cleaning fluorescent lamp adopts open installation. The fluorescent lamp tube wrapped or covered with photo-catalytic coating glass fiber cloth or sleeve is installed on a conventional lamp base seat. When the power of the lamp is turned on, the lamp can provide lighting and air cleaning functions so as to promote air quality. When the lamp is turned on, electric energy is turned into light and heat. The heat causes air convection around the lamp tube outer wall and accelerates waste gas decomposition and adsorption. In some occasions, such as building air condition system, house bathroom venting system, fan for air conditioner, the present invention photo-catalytic air cleaning fluorescent lamp can also be used.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIGS. 1A-1C are schematic illustrations showing the structure of the photo-catalyst coating on the surface of the glass fiber according to the invention;

FIGS. 2A-2C are schematic illustrations showing the process of wrapping a fluorescent lamp with the photo-catalyst coated glass fiber woven cloth according to the invention;

FIGS. 3A-3C are schematic illustrations showing the wrapping of fluorescent lamp having different shapes with the photo-catalyst coated glass fiber cloth according to the invention;

FIGS. 4A and 4B are schematic illustrations showing the wrapping of linear fluorescent lamp with the photo-catalyst coated glass fiber woven cloth according to the invention;

FIGS. 5A-5D are schematic illustrations showing different installation modes of a fluorescent lamp for treating waste gases with the photo-catalyst coated glass fiber sleeve according to the invention;

FIGS. 6A-6C are schematic illustrations showing waste gas decomposition mechanism according to the invention;

FIGS. 7A-7C are schematic illustrations showing an open-type of installation of the fluorescent lamp for treating waste gases according to the invention, and the flowing and diffusion of waste gases under a state of nature convection;

FIGS. 8A and 8B are schematic illustrations showing an open-type of installation of the fluorescent lamp for treating waste gases according to the invention and the flowing and diffusion of waste gases under a state of forced convection;

FIG. 9 shows a Raman spectrum of anatase TiO₂ powder;

FIG. 10 shows Raman spectrum of anatase TiO₂ sol; and

FIG. 11 shows DLS particle size analysis on anatase TiO₂ sol.

### DETAILED DESCRIPTION OF THE PREFERRED EMBODIMENTS

The present invention uses sol-gel technique to prepare anatase TiO2 semiconductor nano crystalline sol, hereinafter referred to as anatase TiO2 sol, which is used for the photocatalytic coating of base materials such as glass, ceramic, carbon materials, metals, plastics, or woven clothes. The coating is first air dried at room temperature, then baked at relatively low temperatures (about 100-250 degrees centigrade). To increase waste gas removal efficiency (or water treatment efficiency), oxidative precious metals or transition metals are added into the prepared anatase TiO₂ sol. Alternatively, the coating may be dipped in solution containing oxidative precious metals ion or transition metals ion, followed by about 100 degrees centigrade drying.

According to the process of the invention, the addition of oxidation catalyst is carried out, after sol-gel coating a photo-catalyst on the fiber woven cloth or sleeve, by impregnating the cloth or sleeve with a solution of oxidation catalytic metal salt. Since the fiber woven cloth or sleeve itself has a meso-pores and the photocatalyst coating has many micro pores, when the photo-catalyst coated fiber cloth or sleeve is dipped in the solution of metal salts, the oxidation catalytic metal salts is adsorbed in the meso pores within the fiber and/or be absorbed in the micro-pores within the photo-catalyst coating, which, after evaporating the solvent, has many fine metal salts remaining on the fiber cloth or sleeve, and thus accomplishes the process of incorporation of oxidation catalysts in the photo-catalyst coated fiber cloth or sleeve.

Under irradiation of visible light and few of UV light, this layer of photo-catalyst coating will generate free electron hole pairs. Oxygen and water on the surface of the catalyst will receive such electron hole pairs and become in a meta-stable state having oxidizing ability. When those precious metal or transition metal ions in a meta-stable state also having oxidizing ability encounter the organic or inorganic gases in the air, a chemical bonding and degradation reaction will take place immediately. Under constant photocatalysis reactions, the hazardous waste gases in the air will be degraded into benign gases which consist mainly of carbon dioxide and water. This photo-catalytic reaction mechanism can be illustrated as follows:

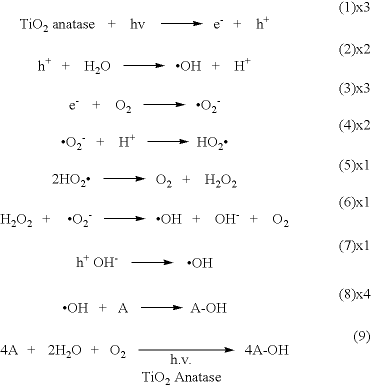

The above-mentioned reaction equations can be balanced as (1)×3+(2)×2+(3)×3+(4)×2+(5)+(6)+(7)+(8)×4=(9). From equation (9), by way of example, when waste gas (A) is reacted firstly with .OH, 4 moles of waste gas require 2 moles of water and one mole of oxygen. Thus, this indicates that the photo-catalytical reaction needs absolutely both water and oxygen. This conclusion is supported by the fact that, in the case of photo-catalytic hydrolysis of organic materials in water, the reaction efficiency in the aqueous solution lack of dissolved oxygen is poor, and likewise, the reaction efficiency in air lack of moisture is also poor. Unless, subsequent to the photo-catalytic degradation of waste gases in air, the product contains water or substances that can react with H⁺ in a manner analogous to water and thereby forms .OH and H⁺, the reaction mechanism can proceed continuously.

After the fluorescent lamp for lighting purposes covered with photo-catalytic cloth, the precious metal or transition metal oxide concentration on anatase TiO₂ particle is below about 1.0% by weight to maintain the maximum brightness or visible light transmission ratio. This is a critical limitation since any anatase TiO₂ photo-catalytic film having precious metal or transition metal oxide concentration to anatase TiO₂ particle exceeding this value will have reduced fluorescent lamp brightness. It is advantageous to use the present invention because that the anatase TiO₂ particle is at nano-scale and has porous structure, resulting in a quantum effect, therefore having photo-catalytic effect when irradiated by visible light. In practice, precious metal or transition metal additives are not so needed.

The photo-catalyst sol used in the above-said process for coating photo-catalyst contains as the main component a titanium alkoxide such as Ti(OR)₄, wherein R is a hydrocarbon group, CₙH₂ₙ₊₁, where n=1−5, and is, for example, methyl, ethyl, n-propyl, isopropyl, n-butyl, t-butyl, sec-butyl, pentyl and the like. Since the anatase TiO₂ particle is stable at about pH 2.5 acid solution and about pH 11.0 alkaline solution, acid-type anatase TiO₂ sol and alkaline-type anatase TiO₂ sol are both developed. To control about 80% of TiO₂ particles to be under an about 100 nm particle size, the anatase TiO₂ sol is incorporated with chelating agents. Inorganic acids or organic acids are used to peptized the gel from hydrolysis and to control the particle size to adjust pH value of the sol. Organic acids include RCOOH. Organic alkali include quandary ammonium R₄NOH and NR₃. Strong chelating agents such as organic acid acetate CH₃C(O)CH₂C(O)R, amino acid RCH(NH₂)COOH, succinic acid HOOCCH(R)COOH, and phenol alcohol RC₆H₃(OCH₃)OH are also used. Amount of strong chelating agent added should be controlled to a chelating agent/Ti(OR)₄ mol ratio of about 0.01-1.0. The use of the chelating agent is before the hydrolysis of the Ti(OR)₄. The chelating agent reacts with the Ti(OR)₄ to form Ti(OR)₄-SCA complex, wherein SCA means Strong Chelating Agent. The complex is then added into water or alcohol-containing aqueous solution to hydrolyze so as to form HₓTiO_{[(3-x)/2+x]}-SCA. Since the mol ratio of chelating agent/Ti(OR)₄ is less than about 1.0, after hydrolysis, the H_{y}TiO_{[(4-y)/2+y]} will mix with the HₓTiO_{[(3-x)/2+x]}-SCA to form a gel. Alternatively, Ti(OR)₄ is added into water to form H_{y}TiO_{[(4-y)/2+y]} gel, then chelating agent is added to form H_{y}TiO_{[(4-y)/2+y]}-SCA gel.

Either the above-said H_{y}TiO_{[(4-y)/2+y]}/HₓTiO_{[(3-x)/2+x]}-SCA mix gel or H_{y}TiO_{[(4-y)/2+y]} gel are hereinafter referred to as TiO₂-SCA gel. To prepare the anatase TiO₂ fine particle sol, acids such as HNO₃, HCl, or HF or bases such as NH₃ or NH₄OH are used to adjust pH value. Acids are used to adjust the sol to about pH 2.5, while the bases are used to adjust the sol to about pH 11.0. After adjusting the pH value, most of the TiO₂ gel begins to peptize, and undergoes rapid peptizing when heated. At this phase, crystalline particles form after the TiO₂ peptizing process. To obtain crystalline TiO₂ particles, the process temperature has to be kept at above about 100 degrees centigrade as hydrothermal process. The resultant anatase TiO₂ particle size relates to the type of chelating agent, chelating agent concentration, dispensing technique when peptizing or dispensing technique in hydrothermal process. It is found that high efficiency dispensing technique can lower the anatase TiO₂ particle size. Higher hydrothermal temperature or longer hydrothermal process results in anatase TiO₂ particle having better crystal structure. Preferably, the hydrothermal temperature is about 250 degrees centigrade. However, it is noted that higher hydrothermal temperature or longer hydrothermal also results in larger crystal size, exceeding 100 nm. The type of chelating agent and its concentration depend on pH value. Proper pH value and hydrothermal temperature are first selected. An about 1 hour to 7 days hydrothermal is preferably carried out to form anatase TiO₂ sol.

In one embodiment, H₄TiO₄ sol contains as binder which is made by the titanium alkoxide such as Ti(OR)₄, wherein R is a hydrocarbon group, CₙH₂ₙ₊₁, where n=1−5, and is, for example, methyl, ethyl, n-propyl, isopropyl, n-butyl, t-butyl, sec-butyl, pentyl and the like. The titanium alkoxide is slowly added into water to form water/titanium alkoxide mol ratio of about 100-1000. The solution is stirred to hydrolyze so as to form the above-said H_{y}TiO_{[(4-y)/2+y]} gel solution. The above-said H_{y}TiO_{[(4-y)/2+y]} gel solution is filtered and washed, and re-filtered to obtain H_{y}TiO_{[(4-y)/2+y]} gel. The thus-formed H_{y}TiO_{[(4-y)/2+y]} gel is then dispensed into water to form water/titanium dioxide mol ratio of 100-1000. After that, the thus-formed H_{y}TiO_{[(4-y)/2+y]} gel solution is cooled down using ice water to below about 4.0 degrees centigrade. Then, an about 33% by weight H₂O₂ solution is added to the cooled H_{y}TiO_{[(4-y)/2+y]} gel solution. The H₂O₂/titanium dioxide mol ratio is about 4.0. The temperature of the H_{y}TiO_{[(4-y)/2+y]} gel solution is kept below about 4.0 degrees centigrade, waiting for the H_{y}TiO_{[(4-y)/2+y]} gel to be completely dissolved into transparent yellow H₄TiO₄ gel. In practice, the concentration of the H₄TiO₄ may be adjusted to about 1.0% by weight and stored in plastic tank at about 4 degrees centigrade to become H₄TiO₄ sol.

When adding the H₄TiO₄ sol into the anatase TiO₂ sol, the H₄TiO₄ to anatase TiO₂ ratio is between about 0-10% by weight. During the addition of the H₄TiO₄ sol, the anatase TiO₂ sol is cooled in iced water at about 4 degrees centigrade. The mixture is then stirred and then stored in a refrigerator at about 4 degrees centigrade. Blending the neutral H₄TiO₄ sol into the anatase TiO₂ sol can increase viscosity of the anatase TiO₂ sol. When coating the glass tube of a fluorescent lamp with the above-said anatase TiO₂ sol mixed with H₄TiO₄ sol, and after an about 100-250 degrees centigrade baking, it is found that the adhesion ability, thickness, and solidity of the coated film are improved, without affecting its porosity.

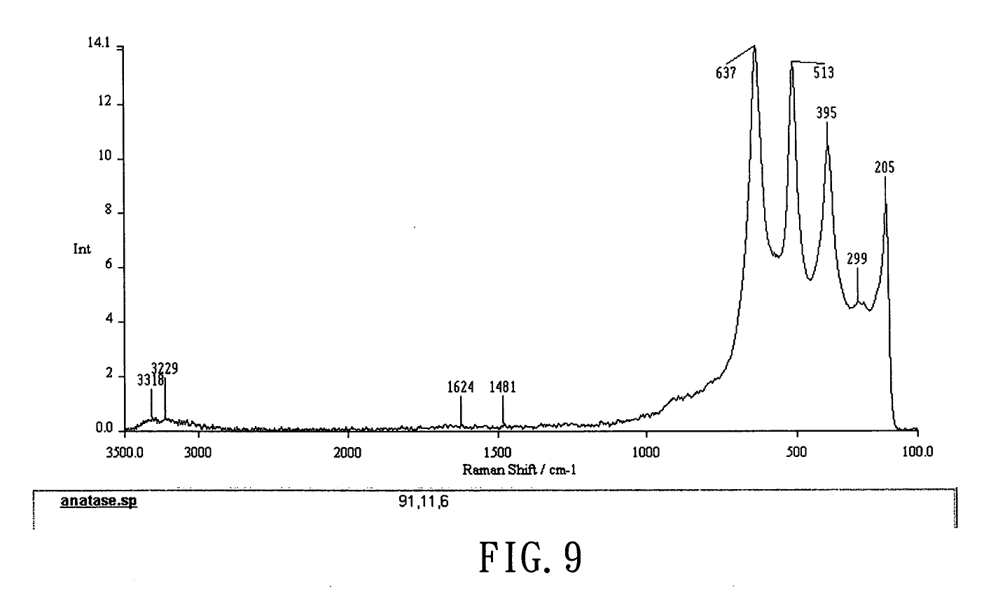

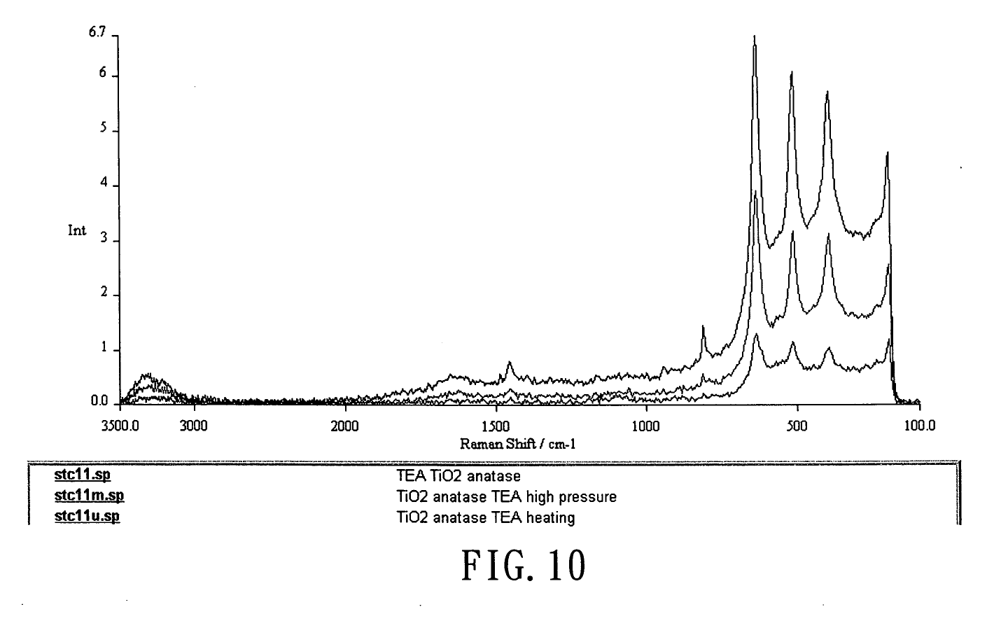

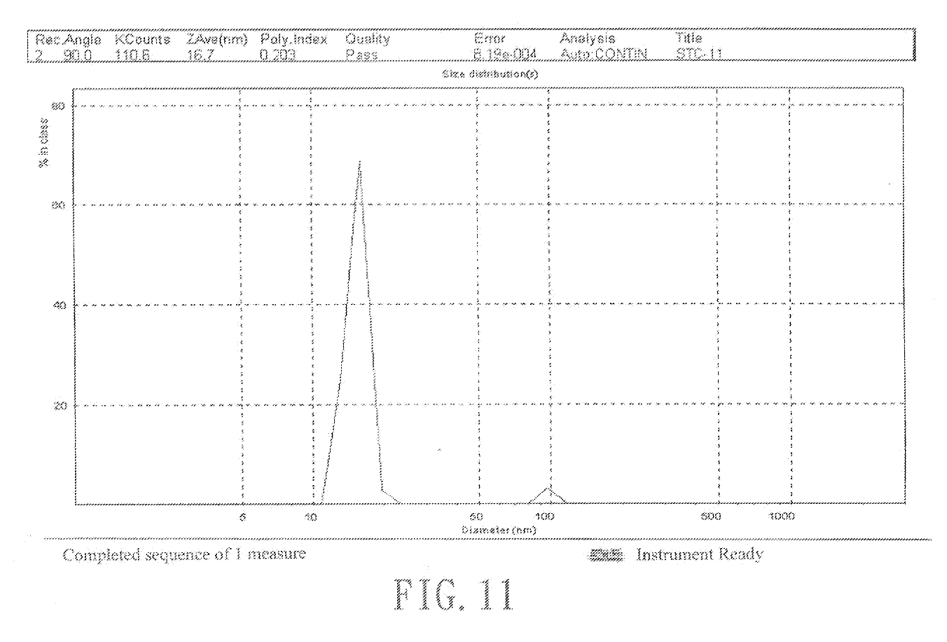

The thus-formed anatase TiO₂ sol is analyzed with Fourier-Transform Raman (FT-Raman) spectroscopy. The resultant Raman shift spectrum is illustrated in FIG. 10. The spectrum is measured by using an about 15% anatase TiO₂ sol, which is irradiated by a 750 mW laser at a wavelength of 1060 nm. As shown in FIG. 10, split high-intensity peaks present at Raman shift 204 cm⁻¹, 398 cm⁻¹, 515 cm⁻¹, and 638 cm⁻¹, which are analogous to the solid anatase TiO₂ Raman shift spectrum as shown in FIG. 9. The particle size is analyzed by DSL laser method. As shown in FIG. 11, the result shows that about 80% of the anatase TiO₂ crystals have a particle size around 10 nm.

The thus-formed TiO₂ sol can be incorporated with other photo-catalytic components including WO₃, ZnO, SnO₂, and Fe₂O₃ which can be added as organic and/or inorganic salts thereof. The inorganic salts thereof can be halides and nitrates, whereas the organic salts can be acetates and acetacetonate provided that they are soluble in the alcohol solvent. The alcohol solution obtained after dissolving completely can be evaporated to remove water and then re-dissolved by adding alcohol solvent to form a precursor alcohol solution of WO₃, ZnO, SnO₂, and Fe₂O₃. Addition of the MOx precursor alcohol solution in desired amount to lead to a weight ratio of MOx/TiO₂=1-100% results in a photo-catalyst coating forming sol.

In order to improve the capacity and efficiency of photo-catalyst coating on treating waste gases, such as those containing organic substances having halogen, nitrogen, phosphorus and sulfur elements, reacted with TiO2 Anatase photo-catalyst by thermal diffusion to doped oxidation catalysts with F, N, P or S components as another type semiconductor for photo-catalyst with lower active energy as visible light for photocatalysis. Suitable oxidation catalysts can be those commonly used, including such as, precious metal type and transition metal type. The precious metal type is usually present in its elemental state, such as, for example, Pd, Pr, Au or Ag, whereas the transition metal type is present as metal oxides such as, for example WO₃, ZnO, SnO₂, Fe₂O₃, MoO₃, Nb₂O₅, V₂O₅, CeO₂ or Cr₂O₃. The amount of such oxidation catalysts in the photocatalyst is in a range of about 0-10.0 wt %. Because such oxidation catalyst itself exhibits an ability of oxidizing waste gases in the air as well as can capture free electrons, electron hole or active radicals generated from the action of the free electrons and electron hole pairs on O₂ and H₂O, such as, .OH, H⁺, .O₂⁻, HO₂., OH.⁻³¹ and the like which are released subsequently for oxidative degrading waste gases as they approach, such that the existing time period of electron hole and free electrons can be sustained and thereby improve the capacity and efficiency of the photo-catalysts even under visible light.

The thus-formed photo-catalyst coating-forming sol can be used then to apply on a substrate such as glass, ceramics, active carbon or metal, which, preferably, are transparent and in fibrous shape. In one embodiment of the invention, the substrate is a fiber or a fiber bundle. The sol-gel coating can be applied directly on the fiber or fiber bundle, and after weaving of the fiber. Since, after sol-gel coating, the fiber and fiber bundle can be bonded directly by an adhesive into a useful non-woven, otherwise, it might be damaged by weaving machine during weave after sol-gel coating. Therefore, it is desirable to apply sol-gel coating on fiber woven cloth and bake the same to fabricate the desired photo-catalyst coated fiber cloth or sleeve.

In order to improve the efficacy of the fluorescent lamp, and to not allow the visible light generated from the fluorescent lamp to be absorbed by opaque materials such that the lighting function of the air cleaning fluorescent lamp cannot be worked. In one embodiment of the invention, quartz or glass fiber materials are used as the substrate. With glass fiber woven cloth or sleeve as a photo-catalytic coating carrier, most visible light transmits through the glass fiber woven cloth or sleeve, and a portion of near UV and blue light are absorbed to act the photocatalytic coating to carry out waste gas decomposition.

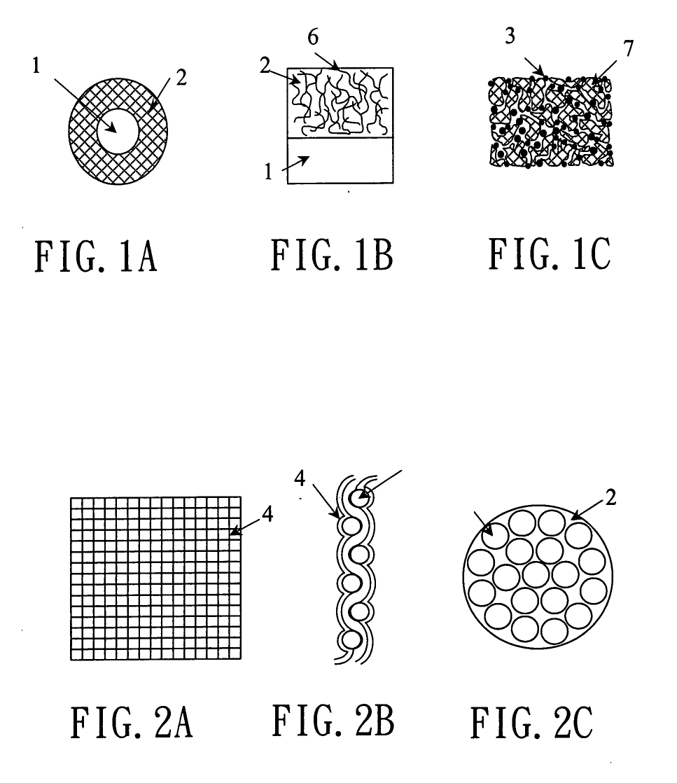

Now, referring to FIGS. 1A-C, the structure of the photo-catalyst thin coating on the surface of the quartz or common glass fiber prepared by the above-described sol-gel coating process according to the invention and impregnated with oxidation catalysts will be illustrated as follow: if a single glass fiber <1> is photo-catalyst coated <2>, as shown in FIG. 1(A), there are tine interstitial pathway <6> surrounding the anatase TiO₂ crystal <7> within the coating, as shown in FIG. 1(B), and a plurality of fine oxidation catalysts <3> are adsorbed on the surface of the coating as well as in the internal interstitial pathway, as shown in FIG. 1(C).

If a bundle consisting a number of glass fibers <5> has been photo-catalyst coated <2>, as shown in FIG. 2(C), similarly, there are likewise anatase TiO₂ crystals <7> and tine interstitial pathways <6> within the structure of the photo-catalyst coating, and there are a plurality of fine oxidation catalysts <3> absorbed on the surface of the coating as well as in the inner interstitial pathways. If a glass fiber woven cloth <4> has been photo-catalyst coated <2>, as shown in FIG. 2(A), a photo-catalyst coated glass fiber woven cloth <41> is obtained, as shown in FIG. 2(B), there are again anatase TiO₂ crystals <7> and tine interstitial pathways <6> within the structure of the photo-catalyst coating, and there are a plurality of fine oxidation catalysts <3> absorbed on the surface of the coating as well as in the inner interstitial pathways.

Now, the fabrication of the fluorescent lamp for air cleaning around the lamp and environment according to the invention will be explained below.

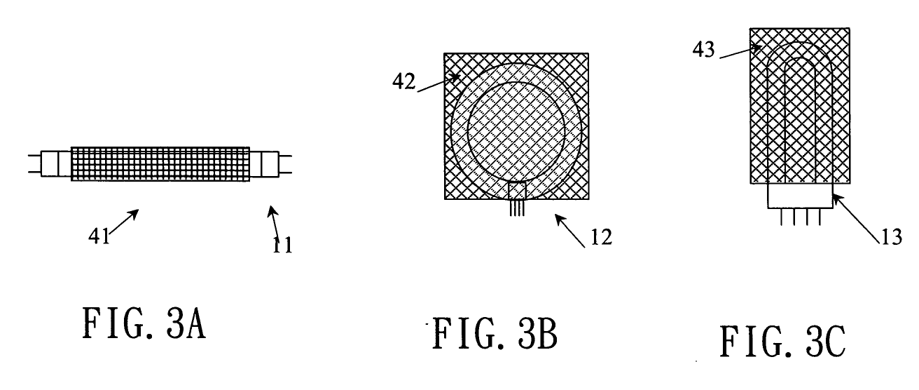

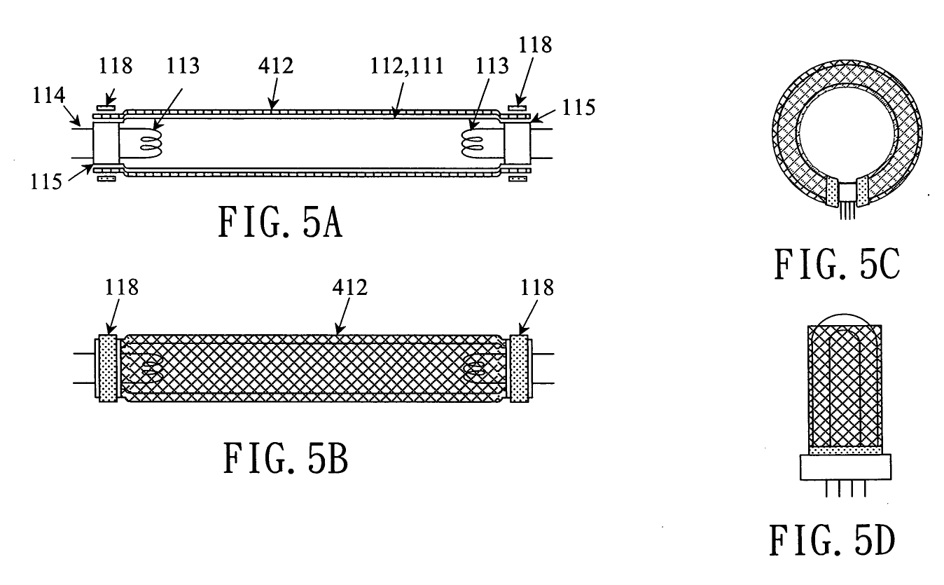

The fluorescent lamp for treating waste gases according to the invention is fabricated by wrapping around a fluorescent lamp tube with a photo-catalyst coated glass fiber woven cloth in a wound-type, covering box-type or sleeve-type, as shown in FIG. 3. In case of using straight fluorescent lamp tube <11>, one or two round of a photo-catalyst coated glass fiber cloth <41> are wound plainly around the tube and fixed on the glass tube by applying on both end and the edge with adhesives such as UV light resistant silicone type adhesives or glass glue, such as shown in FIG. 3(A). The photo-catalyst coated glass fiber sleeve <44> is wound around and fixed on the straight fluorescent lamp by a two-sided adhesive film and then sealed the edge by a thermal melting plastic ring belts, as shown in FIGS. 5(A) and 5(B).

In the case of circular fluorescent lamp tube <12>, the photocatalyst-coated glass fiber cloth can be tailored into a covering box <42> and the box covers the circular fluorescent lamp tube, as shown in FIG. 3(B). The photocatalyst-coated glass fiber sleeve <44> is wound around and fixed on the circular fluorescent lamp by a two-sided adhesive film and then sealed the edge by thermal melting plastic ring belts, as shown in FIGS. 5(C). While in the case of U-shaped fluorescent lamp tube <13>, the photocatalyst-coated glass fiber cloth can be tailored into a sleeve <43> and slip the sleeve <43> on the U-shaped Fluorescent lamp tube, as shown in FIG. 3(C). The photocatalyst-coated glass fiber sleeve <44> is wound around and fixed on the U shaped fluorescent lamp by a two-sided thermal melting plastic ring belt, as shown in FIGS. 5(D).

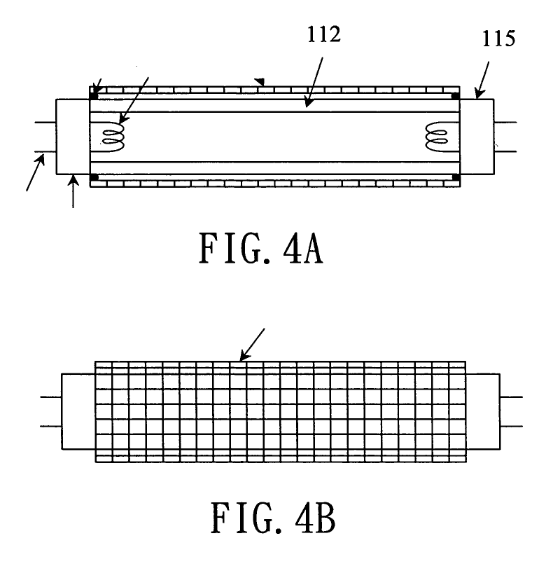

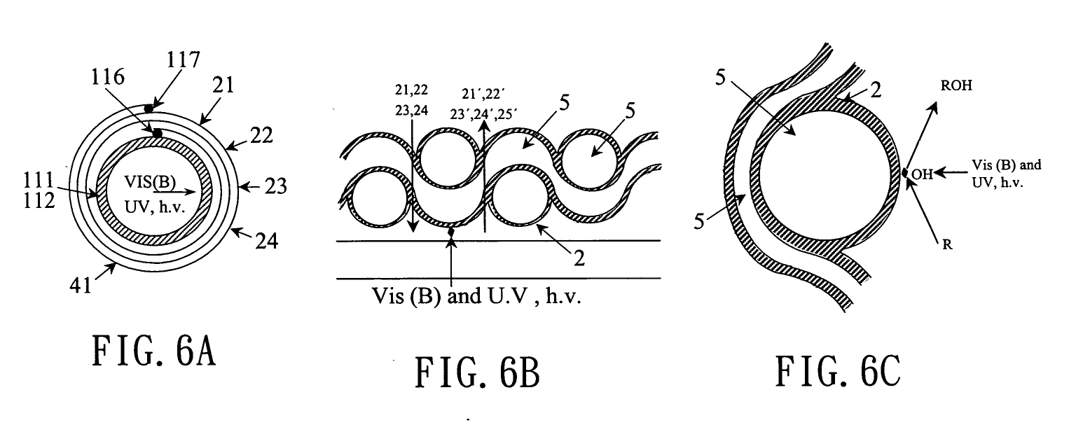

In order to sustain the original function of the fluorescent lamp, the straight fluorescent lamp can be wrapped on whole tube with a photocatalyst-coated glass fiber cloth in a manner as <411> shown in FIG. 4(B) with its cross-section view shown in FIG. 4(A). As to the structure of that fluorescent lamp, a soda lime glass tube <112> is vacuum-sealed at both ends. The heating filaments <113> therein are filled with minor amount of mercury and are connected with external heating pins <114>. Next, the tube is sealed and cemented with aluminum bases <115> at both ends with two connect pins. Finally, the photocatalyst-coated glass fiber cloth <41> is wound around and fixed on the Fluorescent lamp by a two-sided adhesive film <116> and then sealed the edge by a quick-drying UV adhesive <117>, as shown in FIG. 6(A), and thereby accomplishes the fabrication of the Fluorescent lamp for air cleaning according to the invention. Straight fluorescent lamp can be covered by photocatalytic coating glass fiber sleeve <44>, as shown in FIG. 5(A). The photocatalytic coating glass fiber sleeve covering the straight fluorescent lamp is fixed by thermal melting plastic ring belts<118>, as the whole sleeve indicated by <412>.

As described above, the fluorescent lamp for air cleaning according to the invention is constructed by wrapping a photocatalyst-coated glass fiber woven cloth around a fluorescent lamp tube such that, when the fluorescent lamp is turned on in the air, a function of air cleaning occurs accordingly. As such, no mater whether the photocatalyst-coated glass fiber woven cloth is used to warp around a straight fluorescent lamp <11>, a circular fluorescent lamp <12> or a U-shaped fluorescent lamp <13> tube, such function of air cleaning always requires three conditions as following: (1) when turned on, fluorescent light of 420-700 nm visible light and small amount of 365 nm and 405 nm near UV emitted by the fluorescent lamp will transmit through the glass tube and illuminate the photocatalyst coating; (2) there are moisture and photocatalytically degradable waste gases in the air, which can diffuse through the large interstitial pathway within the coated glass fiber woven cloth to the photocatalyst coating illuminated by the fluorescent light; and (3) benign gaseous products generated by photocatalytically degrading waste gases in the air and the air itself can diffuse back through the large interstitial pathway within the coated glass fiber woven cloth into the air.

Now, as a yet another aspect of the invention, a process for air cleaning according to the invention will be described below. In the process for air cleaning according to the invention, the above-described fluorescent lamp for air cleaning is used. As the fluorescent lamp for air cleaning is wrapped with a photocatalyst-coated glass fiber woven cloth, the air <21> that contains organic or inorganic hazardous waste gases <22> normally contains also moisture <23> and carbon dioxide <24>, as illustrated in FIG. 6(A), which can pass from outside of the coated glass fiber woven cloth <41> into the interstitial space between the coated glass fiber cloth and the lamp tube by diffusing through the large interstitial pathway, whereupon, as the fluorescent light emitted by the fluorescent lamp irradiates on the photocatalyst <2>, electron hole pairs generated will combine with O₂ and H₂O in the air to produce .OH free radical which then undergoes a oxidative degradation reaction with such hazardous waste gas <22> in the air according to the reaction equations (1) to (8) and the balanced reaction equation (9). The reaction products comprise H₂O <23>, CO₂ <24> and other gases <25>, which, in combination with some O₂ consumed residual air <21′>, unreacted waste gases <22′>, remaining moisture H₂O <23′>and total CO₂ <24′>, discharge out of the coated glass fiber cloth <41> and sleeve <44> by back diffusing through the large interstitial pathway within said coated glass fiber woven cloth as shown in FIG. 6(B), while the change of reactants and products occurred upon light illuminating the photocatalyst coating <2> on the glass fiber yarn bundle <5> is illustrated in FIG. 6(C).

In one embodiment, the process for air cleaning according to the invention comprises an open-type of use of the fluorescent lamp according to the invention, which, based on the fitting with surrounding facilities, can comprise natural convection and forced convection types, while, based on the manner of installation, can comprise horizontal and perpendicular installation types, that is, in such open types, it is unnecessary that the fluorescent lamp for air cleaning has to be in a closed container and the input of gases to be treated in the container and the output of gaseous products from the container must be conducted by a blower. The fluorescent lamp for air cleaning only needs to be installed, whereby, since, when the fluorescent lamp is turned on for lighting, a heat energy from the heating filaments on both ends transfer to the lamp tube, and, in the course of conversion of electric energy into light with heat energy generated also transfer to the lamp tube, and so that some definite heat energy will radiate from the lamp tube, and thereby provide energy required for nature convection and diffusing the air.

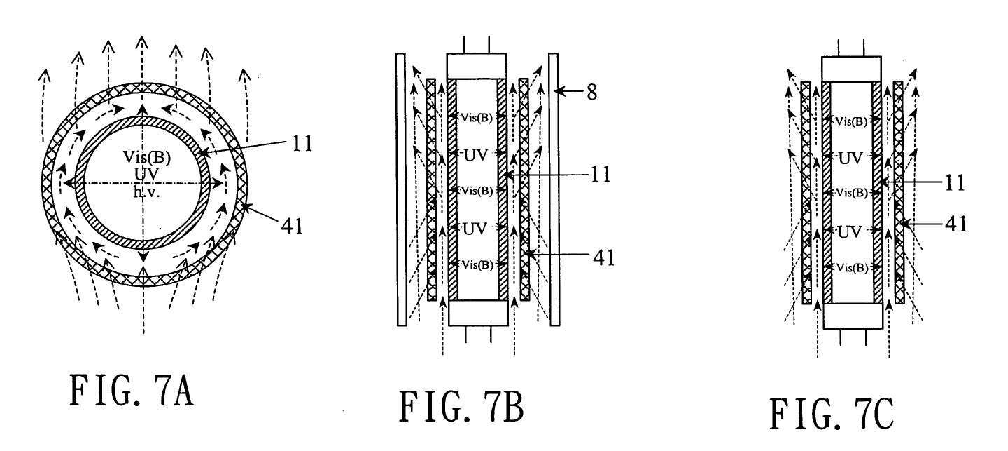

In one embodiment, the fluorescent lamp for air cleaning is hung horizontally, the natural convection of air forces the air <21> beneath the fluorescent lamp to flow upwardly and part of them diffuse into the gap between the photocatalyst-coated glass fiber woven cloth <41> and sleeve <44> and the fluorescent lamp tube, where, after oxidative degradation by the action of the photocatalyst coating and the light, diffuse away the photocatalyst-coated glass fiber cloth <41>, while un-reacted gases diffuses upwardly and outwardly along the gap, and finally, air <21′> in admixture with H₂O <23′>, CO₂ <24′>, residual waste gases <22′> and gaseous reaction products <25> will diffuse upwardly and convection spontaneously away from the fluorescent lamp; meanwhile, gases in the entire space will be continuously treated through gas diffusion and natural convection and by the action of the fluorescent lamp for treating waste gases according to the invention, as illustrated in FIG. 7(A).

In another embodiment, the fluorescent lamp for treating waste gases according to the invention is hung perpendicularly, as shown in FIG. 7(C), where, the diffusion and spontaneous convection of the air, basically, are similar to those occurred in the horizontal installation. However, due to the perpendicular hanging, the natural convection is stronger and the effect of gas diffusion is also stronger, and thereby provides better treating capability for waste gas. In yet another embodiment, an outer sleeve <8> is provided around the fluorescent lamp and results in a better effect as illustrated in FIG. 7(B). Such outer sleeve is made of transparent material and must have an inner diameter larger than that of the fluorescent lamp, for example, an inner diameter twice larger that the outer diameter of the fluorescent lamp, while having a length comparable to that of the fluorescent lamp.

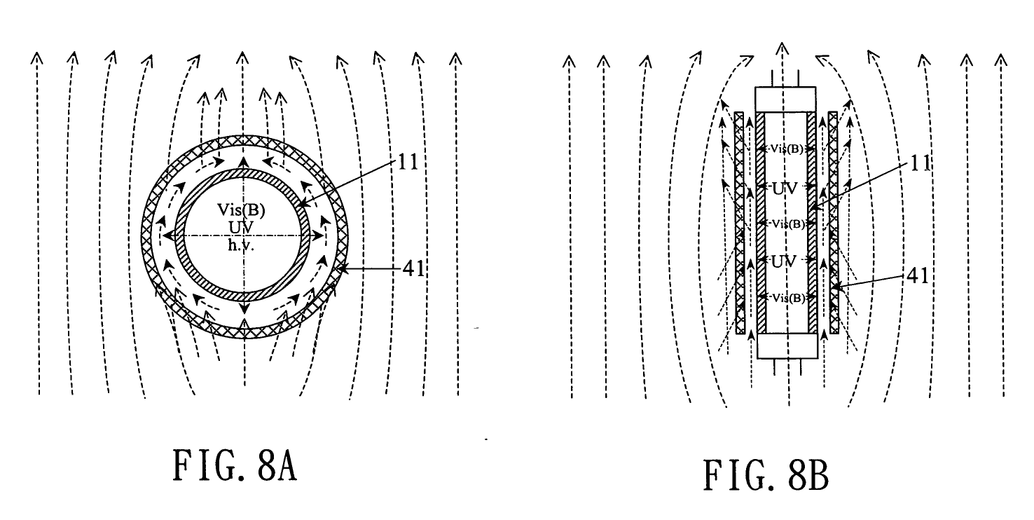

In still another embodiment, in order to arrange a forced air convection, the fluorescent lamp for treating waste gases can be installed in an air flowing space or a conduit with blower, such as, for example, at the outlet of an air conditioner, within the air conduit of an air conditioner, on the base of ventilator in a bathroom, and in a sewer, whereby, the efficiency of air cleaning can be improved by means of external forced air convection, as illustrated in FIGS. 8(A)/(B).

Example for fabricating the present invention fluorescent lamp with photocatalytic coating glass fiber cloth or sleeve, those will be discussed in the following. The fabrication of the fluorescent lamp capable of cleaning air, involves the preparation of the anatase TiO₂ sol and the photocatalytic coating on glass fiber cloth or sleeve for fluorescent lamps. Currently adapted procedure for fabricating the photocatalytic coating fluorescent lamp includes anatase TiO₂ sol dipping and coating the glass fiber cloth or sleeve, followed by 150-250 degree centigrade baking. As mentioned, the thus-formed Anatase TiO₂ sol can be incorporated with other photocatalytic components including WO₃, ZnO, SnO₂, and Fe₂O₃ which can be added as organic and/or inorganic salts thereof. The inorganic salts thereof can be halides and nitrates, whereas the organic salts can be acetates and acetacetonate provided that they are soluble in the alcohol solvent. The alcohol solution obtained after dissolving completely can be evaporated to remove water and then re-dissolved by adding alcohol solvent to form a precursor alcohol solution of WO₃, ZnO, SnO₂, and Fe₂O₃. Addition of the MOx precursor alcohol solution is desired amount to lead to a weight ratio of MOx/TiO2=1-100% results in a photocatalyst coating forming TiO₂ Anatase sol.

The thus-formed photocatalyst coating-forming TiO₂ Anatase sol can then be applied on a substrate such as glass, quartz, which, preferably, are transparent and in fibrous shape. In one embodiment of the invention, the substrate is a fiber or a fiber bundle. The sol-gel coating can be directly applied on the fiber or fiber bundle, or after weaving of the fiber. When applying anatase TiO2 sol mixture on glass fiber cloth and glass sleeve to carry out photocatalytic sol-gel coating, the substrate material is preferably glass or quartz that is transparent to visible light and near UV. The glass fiber cloth and glass sleeve is preferably made of a plurality of single fiber woven or melt into porous, transparent, and in roll form. When applying anatase TiO2 sol mixture on glass fiber cloth and glass sleeve to carry out photocatalytic sol-gel coating, the photocatalyst integrates with the glass fiber cloth and glass sleeve with chemical bonding, such that the photocatalyst will not peel off from the glass fiber cloth and glass sleeve.

In the production of the above-described photocatalyst-coated glass fiber cloth, in order to improve the efficiently of air cleaning, it can be soaked with aqueous solution containing metal salts having oxidative catalytic action. Such metal salts include precious metals as inorganic salts of Pd, Pt, Au and Ag or inorganic salts of transition metals as Mo, Nb, V, Ce or Cr. The glass fiber cloth is ready for use after being soaked with oxidative catalyst and dried. The concentration of the oxidative catalyst precious metal adhesion quantity on the anatase TiO2 coating film is larger than about 0.1 wt %, the nano metals will significantly absorb visible light and thus decrease fluorescent lamp illumination efficiency.

The thus formed photocatalytic coating glass fiber cloth and glass fiber sleeve covering the fluorescent lamp tube can be tailored into the shape of a lamp tube. The above-said anatase TiO₂ sol photocatalytic coating glass fiber cloth or sleeve can be cut into desired size to wrap outside the fluorescent lamp. The cut size depends on lamp length and layers when wrapping the lamp. After covering the lamp with the coated cloth, the ends and/or edges of the wrapping cloth is fixed by UV resistant glue, or fixed by sawing or laser sintering. When using a longitudinally extended outer sleeve covering the fluorescent lamp, the outer sleeve has an inner diameter larger than an outer diameter of the fluorescent lamp tube. The outer sleeve has a length substantially equal to a length of the fluorescent lamp tube. The outer sleeve has opposing open ends that are sealed with thermal melting plastic ring belts to fix on the lamp. The present invention adopts various fluorescent lamps having a fluorescent visible light wavelength of 420-700 nm and small amount of 365 nm and 405 nm near UV, thereby forming an air cleaning fluorescent lamp, which provide lighting and air cleaning functions.

### EXAMPLE 1

In accordance with this preferred embodiment of the present invention, a 4 wt % acidic anatase TiO₂ sol prepared by above-said process is used to coat glass fiber. The coated glass fiber is tailored and woven into sleeve form of lamp tube size. The thus formed glass fiber sleeve is fixed on the fluorescent lamp with thermal glue. The fluorescent lamps include 38 W-DEX and 32 W-DBL. The decomposition efficiency of the above-said fluorescent lamps regarding organic substance butyl acetate is measured in a 5-liter closed chamber system. 5.0 mL butyl acetate is injected into the 5-liter closed chamber system and measured by FTIR during the irradiation of fluorescent lamps. According to the experimental results, the 38 W-DEX fluorescent lamp covered with acidic 4 wt % anatase TiO₂ sol coating photocatalytic glass fiber sleeve has a butyl acetate decomposition rate of 0.120 min⁻¹. The 32 W-DBL fluorescent lamp covered with acidic about 4 wt % anatase TiO₂ sol coating photocatalytic glass fiber sleeve has a butyl acetate decomposition rate of about 0.2567 min⁻¹.

### EXAMPLE 2

In accordance with this preferred embodiment of the present invention, an about 15 wt % alkaline anatase TiO₂ sol prepared by above-said process is used to coat glass fiber. The coated glass fiber is tailored and woven into sleeve form of lamp tube size. The thus formed glass fiber sleeve is fixed on the fluorescent lamp with thermal melting plastic ring belts. The fluorescent lamps include 38 W-DEX and 32 W-DBL. The decomposition efficiency of the above-said fluorescent lamps regarding organic substance butyl acetate is measured in a 5-liter closed chamber system. 5.0 mL butyl acetate is injected into the 5-liter closed chamber system and measured by FTIR during the irradiation of fluorescent lamps. According to the experimental results, the 38 W-DEX fluorescent lamp covered with such anatase TiO₂ sol coating photocatalytic glass fiber sleeve has a butyl acetate decomposition rate of about 0.1581 min⁻¹. The 32 W-DBL fluorescent lamp covered with such anatase TiO₂ sol coating photocatalytic glass fiber sleeve has a butyl acetate decomposition rate of about 0.2765 min⁻¹.

To sum up, the present invention provides methods for preparing nano-scale semiconductor crystalline anatase TiO₂ sol, which is used to coat glass fiber cloth or sleeve for various fluorescent lamps by using the above-mentioned dip coating method. The coated clothes are baked to form photocatalytic coating cloth capable of cleaning air and self-cleaning. The photocatalytic fluorescent lamps can maintain the brightness and illumination. Since the porous characteristic of the anatase TiO₂ coating and due to its visible light photocatalytic ability, the small amount of UV light (UVA) and visible light are absorbed by the anatase TiO₂ coating and thus generating active species such as electron-hole pairs that are capable of air cleaning or purifying.

Various types of fluorescent lamps may be used to incorporate the present invention recipe and process thereof. The Anatase TiO₂ sol either single component anatase TiO₂ sol or multi component anatase TiO₂ sol mixture (comprising TiO₂, WO₃, ZnO, SnO₂, or Fe₂O₃), or anatase TiO₂ sol blended with nano precious metals or nano transition metals oxide may be used to coat glass fiber clothes or sleeve, which is then used to cover the fluorescent lamp tube.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A process for treating waste gases, comprising the steps of:
using a photocatalytic fluorescent lamp capable of cleaning air, the photocatalytic fluorescent lamp device being fabricated by:
formulating a photocatalyst anatase TiO₂ sol mixture and dip coating a glass fiber cloth or glass fiber sleeve with said photocatalyst anatase TiO₂ sol mixture;
drying said photocatalyst sol coated glass fiber cloth or glass fiber sleeve into a photocatalyst-coated glass fiber cloth or glass fiber sleeve, in a first drying step;
impregnating said photocatalyst-coated glass fiber cloth or glass fiber sleeve with a solution of an oxidation catalyst having precious metals or transition metal-oxides;
drying said impregnated photocatalyst-coated glass fiber cloth or glass fiber sleeve again, in a second drying step;
tailoring the photocatalyst sol coated glass fiber or glass fiber sleeve obtained from the first drying step or said impregnated photocatalyst-coated glass fiber cloth or glass fiber sleeve from the second drying step to a fluorescent lamp tube and encompassing at least a portion of said fluorescent lamp tube with said photocatalyst-coated glass fiber cloth or glass fiber sleeve; and
using UV resistant glue, a thermal plastic ring belt, sewing, or laser sintering techniques to fix said photocatalyst-coated glass fiber cloth or glass fiber sleeve on said fluorescent lamp tube;
employing an open type of natural convection, whereby heat energy radiated from the fluorescent lamp heats air adjacent thereto and causes a natural convection of waste gases;
diffusing said waste gases through interstitial spaces within the impregnated photocatalyst-coated glass fiber cloth or glass fiber sleeve into a gap between said fluorescent lamp tube and said impregnated photocatalyst-coated glass fiber cloth or glass fiber sleeve; and
photocatalytically degrading and oxidizing said waste gases and then diffusing said waste gases by natural convection back through said interstitial spaces within said impregnated photocatalyst-coated glass fiber cloth away from said fluorescent lamp tube,

2. The process for treating waste gases of claim 1, further comprising the steps of:
hanging said fluorescent lamp perpendicularly or horizontally; and
using natural convection of air to force air beneath said fluorescent lamp to flow upwardly so that a portion thereof diffuses into the gap between said photocatalyst-coated glass fiber cloth or photocatalyst-coated glass fiber sleeve and said fluorescent lamp tube,
wherein, when hung perpendicularly, an outer sleeve is provided around said fluorescent lamp, said outer sleeve being made of a transparent material and having an inner diameter twice the size of an outer diameter of said fluorescent lamp and having a length comparable to that of said fluorescent lamp.

3. The process for treating waste gases of claim 1, wherein said fluorescent lamp is installed in an outer sleeve connected to said fan or blower.

4. The process of claim 3, wherein said outer sleeve is made of transparent material and has an inner diameter twice the size of an outer diameter of said fluorescent lamp and a length comparable to that of said fluorescent lamp.
